(19) United States
(12) Patent Application Publication
(10) Pub. No.: US 2012/0009225 A1
(43) Pub. Date: Jan. 12, 2012

(54) Stable Solid Formulation of Therapeutic Polypeptides Suitable for Oral Administration

(75) Inventors: Angelika Fretzen, Somerville, MA (US); Steven Witowski, Melrose, MA (US); Alfredo Grossi, Somerville, MA (US); Hong Zhao, Somerville, MA (US)

(73) Assignee: Ironwood Pharmaceuticals, Inc., Cambridge, MA (US)

(21) Appl. No.: 13/062,332

(22) PCT Filed: Aug. 14, 2009
(86) PCT No.: PCT/US2009/004676

§ 371 Date: Sep. 29, 2011

Related U.S. Application Data
(60) Provisional application No. 61/094,370, filed on Sep. 4, 2008.

(51) Int. Cl. A61K 9/14; A61K 9/00; A61K 9/48; A61K 38/02

(52) U.S. Cl. 424/400; 514/1.1; 427/2.14

(57) ABSTRACT

Solid, stable formulations of therapeutic polypeptide suitable for oral administration are described herein as are methods for preparing such formulations. The therapeutic polypeptide formulations described herein are stable and have a sufficient shelf life for manufacturing, storing and distributing the drug.

### PRIORITY CLAIM

This application claims priority to U.S. Application Ser. No. 61/094,370, filed Sep. 4, 2008. The entire contents of the aforementioned application are incorporated herein by reference.

### FIELD

This disclosure concerns solid formulations of therapeutic polypeptides suitable for oral administration and methods for preparing such formulations.

### BACKGROUND

Many therapeutic polypeptides are formulated in aqueous solution because they are most active in this form. However, most polypeptides are not particularly stable in aqueous solution, such that the formulations often have a short half-life and require refrigeration. Although aqueous solutions of polypeptides can be dried by freeze-drying, spray-drying or other methods, such dried formulations may also be unstable and have reduced activity relative to an aqueous solution of the polypeptide. Typical break-down mechanisms that occur both in aqueous solution and in dried formulations include aggregation and oxidative or hydrolytic degradation. Thus, the majority of therapeutic polypeptides, whether in aqueous solution or dried, are stored under refrigerated conditions due to their limited stability.

### SUMMARY

Solid, stable formulations of therapeutic polypeptides are described herein as are methods for preparing such formulations. The formulations described herein contain a therapeutic polypeptide.

The therapeutic polypeptide formulations described herein can be stable and can have a sufficient shelf life for manufacturing, storing and distributing the drug. For example, formulations described herein are expected to have a shelf life of at least 12 months at room temperature storage conditions (e.g., 25° C./60% relative humidity (RH)). In further embodiments, the formulations described herein are expected to have a shelf life of at least 18 months or at least 24 months at room temperature storage conditions (e.g., 25° C./60% RH). Thus, when assessed in an assay on a weight/weight basis as determined by high pressure liquid chromatography (HPLC) against a therapeutic polypeptide reference standard, ≧95% of the original amount of therapeutic polypeptide in the composition remains after three months when packaged samples are stored at accelerated conditions (40° C./75% RH). In further embodiments, ≧90% of the original amount of therapeutic polypeptide in the composition remains after at least 6 months when packaged samples are stored at accelerated conditions (40° C./75% RH). In addition, chromatographic purity of the therapeutic polypeptide as determined as area percent by HPLC remains at ≧95% over the course of at least three months when packaged samples are stored at accelerated conditions (40° C./75% RH). In further embodiments, the chromatographic purity of the therapeutic polypeptide as determined by area percent by HPLC remains at ≧90% over the course of at least 6 months when packaged samples are stored at accelerated conditions (40° C./75% RH). Thus, for example, no more than about 10% of the therapeutic polypeptide undergoes degradation to other products.

In one embodiment, the invention comprises a pharmaceutical composition comprising therapeutic polypeptide, wherein the chromatographic purity of the therapeutic polypeptide decreases by less than 10% after 18 months or 24 months of storage of the pharmaceutical composition at 25° C. at 60% relative humidity in a sealed container containing a desiccant. In a further embodiment, the chromatographic purity of the therapeutic polypeptide decreases by less than 9%, 8%, 7%, 6%, 5%, 4% or 2% after 18 months or 24 months of storage of the pharmaceutical composition at 25° C. at 60% relative humidity in a sealed container containing a desiccant. In another embodiment, the invention comprises a pharmaceutical composition comprising therapeutic polypeptide, wherein the chromatographic purity of the therapeutic polypeptide decreases by less than 10% after 3 months or 6 months of storage of the pharmaceutical composition at 40° C. at 75% relative humidity in a sealed container containing a desiccant. In a further embodiment, the chromatographic purity of the therapeutic polypeptide decreases by less than 9%, 8%, 7%, 6%, 5%, 4% or 2% after 3 months or 6 months of storage of the pharmaceutical composition at 40° C. at 75% relative humidity in a sealed container containing a desiccant.

In one embodiment, the invention comprises a unit dosage form of a pharmaceutical composition comprising therapeutic polypeptide, wherein the chromatographic purity of the therapeutic polypeptide decreases by less than 10% after 18 months or 24 months of storage of the unit dosage form at 25° C. at 60% relative humidity in a sealed container containing a desiccant. In a further embodiment, the chromatographic purity of the therapeutic polypeptide decreases by less than 9%, 8%, 7%, 6%, 5%, 4% or 2% after 18 months or 24 months of storage of the unit dosage form at 25° C. at 60% relative humidity in a sealed container containing a desiccant. In another embodiment, the invention comprises a unit dosage form of a pharmaceutical composition comprising therapeutic polypeptide, wherein the chromatographic purity of the therapeutic polypeptide decreases by less than 10% after 3 months or 6 months of storage of the unit dosage form at 40° C. at 75% relative humidity in a sealed container containing a desiccant. In a further embodiment, the chromatographic purity of the therapeutic polypeptide decreases by less than 9%, 8%, 7%, 6%, 5%, 4% or 2% after 3 months or 6 months of storage of the unit dosage form at 40° C. at 75% relative humidity in a sealed container containing a desiccant.

In one embodiment, the invention comprises a sealed container comprising a plurality of unit dosage forms of a pharmaceutical composition comprising therapeutic polypeptide, wherein the chromatographic purity of the therapeutic polypeptide decreases by less than 10% after 18 months or 24 months of storage of the sealed container containing a desiccant at 25° C. at 60% relative humidity. In a further embodiment, the chromatographic purity of the therapeutic polypeptide decreases by less than 9%, 8%, 7%, 6%, 5%, 4% or 2% after 18 months or 24 months of storage of the sealed container containing a desiccant at 25° C. at 60% relative humidity. In another embodiment, the invention comprises a sealed container comprising a plurality of unit dosage forms of a pharmaceutical composition comprising therapeutic polypeptide, wherein the chromatographic purity of the therapeutic polypeptide decreases by less than 10% after 3 months or 6 months of storage of the sealed container containing a desiccant at 40° C. at 75% relative humidity. In a further embodiment, the chromatographic purity of the therapeutic polypeptide decreases by less than 9%, 8%, 7%, 6%, 5%, 4% or 2% after 3 months or 6 months of storage of the sealed container containing a desiccant at 40° C. at 75% relative humidity.

In one embodiment, the invention comprises a pharmaceutical composition comprising therapeutic polypeptide, wherein the assay value for therapeutic polypeptide determined on a weight/weight basis decreases by less than 10% after 18 months or 24 months of storage of the pharmaceutical composition at 25° C. at 60% relative humidity in a sealed container containing a desiccant. In a further embodiment, the assay value for therapeutic polypeptide determined on a weight/weight basis decreases by less than 9%, 8%, 7%, 6%, 5%, 4%, 3%, 2% or 1% after 18 months or 24 months of storage of the pharmaceutical composition at 25° C. at 60% relative humidity in a sealed container containing a desiccant. In another embodiment, the invention comprises a pharmaceutical composition comprising therapeutic polypeptide, wherein the assay value for therapeutic polypeptide determined on a weight/weight basis decreases by less than 10% after 3 months or 6 months of storage of the pharmaceutical composition at 40° C. at 75% relative humidity in a sealed container containing a desiccant. In a further embodiment, the chromatographic purity of the therapeutic polypeptide decreases by less than 9%, 8%, 7%, 6%, 5%, 4%, 3%, 2% or 1% after 3 months or 6 months of storage of the pharmaceutical composition at 40° C. at 75% relative humidity in a sealed container containing a desiccant.

In one embodiment, the invention comprises a unit dosage form of a pharmaceutical composition comprising therapeutic polypeptide, wherein the assay value for therapeutic polypeptide determined on a weight/weight basis decreases by less than 10% after 18 months or 24 months of storage of the unit dosage form at 25° C. at 60% relative humidity in a sealed container containing a desiccant. In a further embodiment, the assay value for therapeutic polypeptide determined on a weight/weight basis decreases by less than 9%, 8%, 7%, 6%, 5%, 4%, 3%, 2% or 1% after 18 months or 24 months of storage of the unit dosage form at 25° C. at 60% relative humidity in a sealed container containing a desiccant. In another embodiment, the invention comprises a unit dosage form of a pharmaceutical composition comprising therapeutic polypeptide, wherein the assay value for therapeutic polypeptide determined on a weight/weight basis decreases by less than 10% after 3 months or 6 months of storage of the unit dosage form at 40° C. at 75% relative humidity in a sealed container containing a desiccant. In a further embodiment, the assay value for therapeutic polypeptide determined on a weight/weight basis decreases by less than 9%, 8%, 7%, 6%, 5%, 4%, 3%, 2% or 1% after 3 months or 6 months of storage of the unit dosage form at 40° C. at 75% relative humidity in a sealed container containing a desiccant.

In one embodiment, the invention comprises a sealed container comprising a plurality of unit dosage forms of a pharmaceutical composition comprising therapeutic polypeptide, wherein the assay value for therapeutic polypeptide determined on a weight/weight basis decreases by less than 10% after 18 months or 24 months of storage of the sealed container at 25° C. at 60% relative humidity in a sealed container containing a desiccant. In a further embodiment, the assay value for therapeutic polypeptide determined on a weight/weight basis decreases by less than 9%, 8%, 7%, 6%, 5%, 4%, 3%, 2% or 1% after 18 months or 24 months of storage of the sealed container containing a desiccant at 25° C. at 60% relative humidity. In another embodiment, the invention comprises a sealed container comprising a plurality of unit dosage forms of a pharmaceutical composition comprising therapeutic polypeptide, wherein the assay value for therapeutic polypeptide determined on a weight/weight basis decreases by less than 10% after 3 months or 6 months of storage of the sealed container containing a desiccant at 40° C. at 75% relative humidity. In a further embodiment, the assay value for therapeutic polypeptide determined on a weight/weight basis decreases by less than 9%, 8%, 7%, 6%, 5%, 4%, 3%, 2% or 1% after 3 months or 6 months of storage of the sealed container containing a desiccant at 40° C. at 75% relative humidity.

The assay value on a weight/weight basis (“weight/weight assay”) may be determined by comparing, e.g., by HPLC, the amount of therapeutic polypeptide in a sample, to a therapeutic polypeptide reference standard. As used herein, the weight of therapeutic polypeptide in a composition after storage at room temperature or accelerated conditions at a specified time point (e.g., three or six months of storage under accelerated conditions [40° C./75% RH] or 12, 18 or 24 months of storage under room temperature conditions [25° C./60% RH]) is compared to the weight of therapeutic polypeptide in a composition at an initial time (e.g., the time when the pharmaceutical composition is released for clinical or patient use (“the release date”)) to provide the weight/weight assay value. For example, the weight of therapeutic polypeptide in a composition is measured after storage for a specified time at accelerated conditions (40° C./75% RH) and compared to the weight of therapeutic polypeptide that was present in the sample at the release date. In another example, the weight of therapeutic polypeptide in a composition is measured after storage for a specified time at room temperature conditions (25° C./60% RH) and compared to the weight of therapeutic polypeptide that was present in the sample at the release date. Thus, the phrase “≧90% of the original amount of therapeutic polypeptide in the composition remains after at least 6 months when packaged samples are stored at accelerated conditions (40° C./75% RH)” means the weight of therapeutic polypeptide in the composition measured in an assay on a weight/weight basis as determined by HPLC after at least 6 months storage at accelerated conditions is ≧90% of the amount of therapeutic polypeptide in the composition present at the initial time (e.g., the release date of the therapeutic polypeptide composition).

Chromatographic purity of therapeutic polypeptide may be assessed by performing HPLC under the conditions described herein. The area under the therapeutic polypeptide peak is measured and compared to the total area under all peaks excluding the solvent peak and any non-polypeptide related peaks (i.e., peaks associated with excipients that may be observed in a placebo). As used herein, the chromatographic purity of therapeutic polypeptide in a composition after storage at room temperature or accelerated conditions at a specified time point (e.g., three or six months of storage under accelerated conditions [40° C./75% RH] or 12, 18 or 24 months of storage under room temperature conditions [25° C./60% RH]) is compared to the chromatographic purity of therapeutic polypeptide in a composition at an initial time (e.g., the time when the pharmaceutical composition is released for clinical or patient use (“the release date”)) to provide the chromatographic purity value. For example, the chromatographic purity of therapeutic polypeptide in a composition is measured after storage for a specified time at accelerated conditions (40° C./75% RH) and compared to the chromatographic purity of therapeutic polypeptide in the composition at the release date. In another example, the chromatographic purity of therapeutic polypeptide in a composition is measured after storage for a specified time at room temperature conditions (25° C./60% RH) and compared to the chromatographic purity of therapeutic polypeptide in the composition at the release date.

This disclosure features a method for preparing a pharmaceutical composition comprising therapeutic polypeptide or a pharmaceutically acceptable salt thereof, the method comprising: (a) providing a solution, e.g., an aqueous solution (“the coating solution”), comprising: (i) purified therapeutic polypeptide or a pharmaceutically acceptable salt thereof; (ii) a cation selected from Mg²⁺, Ca²⁺, Zn²⁺, Mn²⁺, K⁺, Na⁺ or Al³⁺and/or a sterically hindered primary amine (e.g., leucine) and, optionally, (iii) a pharmaceutically acceptable binder; and (b) applying the coating solution to a pharmaceutically acceptable filler to generate polypeptide-coated filler (e.g., by spraying, mixing or coating the pharmaceutically acceptable filler with the coating solution). The method can optionally include one or more of: (i) blending the polypeptide-coated filler with a pharmaceutically acceptable glidant, a pharmaceutically acceptable lubricant or a pharmaceutically acceptable additive that acts as both a glidant and lubricant; (ii) blending the polypeptide-coated filler with filler that is not polypeptide-coated, (iii) blending the polypeptide-coated filler with other additives; (iii) applying a pharmaceutically acceptable coating additive to the polypeptide-coated filler. The final pharmaceutical composition can be placed into capsules (e.g., gelatin capsule) or used to form tablets.

In some embodiments, there is provided a pharmaceutical composition comprising a pharmaceutically acceptable carrier, therapeutic polypeptide and one or more agents selected from Mg²⁺, Ca²⁺, Zn²⁺, Mn²⁺, K⁺, Na⁺ or Al³⁺and a sterically hindered primary amine, wherein the agent improves at least one attribute of the composition, relative to a pharmaceutical composition without the agent. In further embodiments, the agent is Mg²⁺, Ca²⁺or Zn²⁺. In a further embodiment, the agent is Ca²⁺. In another embodiment, the agent is a sterically hindered primary amine. In a further embodiment, the sterically hindered primary amine is an amino acid. In yet a further embodiment, the amino acid is a naturally-occurring amino acid. In a still further embodiment, the naturally-occurring amino acid is selected from the group consisting of: histidine, phenylalanine, alanine, glutamic acid, aspartic acid, glutamine, leucine, methionine, asparagine, tyrosine, threonine, isoleucine, tryptophan, methionine, glycine, and valine; yet further, the naturally-occurring amino acid is leucine, isoleucine, alanine or methionine; in another embodiment, the naturally-occurring amino acid is leucine or methionine; still further, the naturally-occurring amino acid is leucine. In another embodiment, the sterically hindered primary amine is a non-naturally occurring amino acid (e.g., 1-aminocyclohexane carboxylic acid). In a further embodiment, the sterically hindered primary amine is cyclohexylamine, 2-methylbutylamine or chitosan. In another embodiment, the sterically hindered primary amine can be a mixture of more than one sterically hindered primary amine. For example, the sterically hindered primary amine may be a mixture of two or more amino acids. In further embodiments, the pharmaceutical composition comprising a therapeutic polypeptide is a mixture of two or more therapeutic polypeptides.

In other embodiments, there is provided a pharmaceutical composition comprising a pharmaceutically acceptable carrier, therapeutic polypeptide, a cation selected from Mg²⁺, Ca²⁺, Zn²⁺, Mn²⁺, K⁺, Na⁺or Al³⁺and a sterically hindered primary amine. In one embodiment, the cation is Ca²⁺. In another embodiment, the cation is a mixture of two or three of Mg²⁺, Ca²⁺and Zn²⁺. In a further embodiment, the pharmaceutical composition further comprises a pharmaceutically acceptable binder and/or a pharmaceutically acceptable glidant, lubricant or additive that acts as both a glidant and lubricant and/or an antioxidant. In a further embodiment, the sterically hindered primary amine is an amino acid. In yet a further embodiment, the amino acid is a naturally-occurring amino acid. In a still further embodiment, the naturally-occurring amino acid is selected from the group consisting of: histidine, phenylalanine, alanine, glutamic acid, aspartic acid, glutamine, leucine, methionine, asparagine, tyrosine, threonine, isoleucine, tryptophan, methionine, glycine, and valine; yet further, the naturally-occurring amino acid is leucine, isoleucine, alanine or methionine; in another embodiment, the naturally-occurring amino acid is leucine or methionine; still further, the naturally-occurring amino acid is leucine. In another embodiment, the sterically hindered primary amine can be a mixture of more than one sterically hindered primary amines. For example, the sterically hindered primary amine may be a mixture of two or more amino acids.

In some cases the molar ratio of cation:sterically hindered primary amine:therapeutic polypeptide (e.g., Ca²⁺:leucine:therapeutic polypeptide) in the aqueous solution applied to the carrier is 5-100:5-50:1. It can be desirable for the molar ratio of cation:sterically hindered primary amine (e.g., Ca²⁺:leucine) to be equal to or greater than 2:1 (e.g., between 5:1 and 2:1). Thus, in some cases the molar ratio of cation:sterically hindered primary amine:therapeutic polypeptide (e.g., Ca²⁺:leucine:therapeutic polypeptide) applied to the carrier is 100:50:1, 100:30:1, 80:40:1, 80:30:1, 80:20:1, 60:30:1, 60:20:1, 50:30:1, 50:20:1, 40:20:1, 20:20:1, 10:10:1, 10:5:1 or 5:10:1. When binder, e.g., methylcellulose, is present in the therapeutic polypeptide solution applied to the carrier it can be present at 0.5%-2.5% by weight (e.g., 0.7%-1.7% or 0.7%-1% or 1.5% or 0.7%).

The weight of therapeutic polypeptide applied to a given weight of filler (e.g., microcrystalline cellulose) can vary from about 0.02:100 to about 2.67:100. Thus, about 0.05 mg to about 6.0 mg of therapeutic polypeptide can be applied to 225 mg of filler. In a further embodiment, the weight of therapeutic polypeptide applied to a given weight of filler is about 0.05 mg to about 2.0 mg of therapeutic polypeptide (e.g., 0.1, 0.2, 0.3. 0.4, 0.5, 0.6, 0.7 mg peptide for 225 mg of filler).

In various embodiments: the sterically hindered primary amine is an amino acid (e.g., a naturally-occurring amino acid or a naturally-occurring amino acid selected from histidine, phenylalanine, alanine, glutamic acid, aspartic acid, glutamine, methionine, asparagine, tyrosine, threonine, leucine, isoleucine, tryptophan, glycine or valine). In other cases the sterically hindered primary amine is a non-naturally occurring amino acid (e.g., lanthionine, theanine or 1-aminocyclohexane carboxylic acid). In a further embodiment, the sterically hindered primary amine is cyclohexylamine or 2-methylbutylamine. In other cases, the sterically hindered primary amine is an amino sugar (e.g., chitosan or glucosamine).

In some cases, the sterically hindered primary amine has the formula:

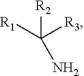

wherein R₁, R₂ and R₃ are independently selected from: H; —C(O)OH; C₁-C₆ alkyl, optionally substituted by —CO₂H, —CONH₂, or a 5-10 membered aryl or heteroaryl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

In various cases: the antioxidant is selected from BHA (butylated hydroxyanisole), BHT (butylated hydroxytoluene), vitamin E, propyl gallate, ascorbic acid and salts or esters thereof, tocopherol and esters thereof, alpha-lipoic acid, beta-carotene; the pharmaceutically acceptable binder is polyvinyl alcohol; the pharmaceutically acceptable binder is selected from: a starch (e.g., corn starch, pre-gelatinized potato starch, rice starch, wheat starch, and sodium starch glycollate), maltodextrin and a cellulose ether (e.g., methyl cellulose, hydroxyethyl cellulose, hydroxyethyl methyl cellulose and hydroxypropyl methyl cellulose); the pharmaceutically acceptable filler is cellulose (e.g., microfine cellulose or microcrystalline cellulose); the pharmaceutically acceptable filler is a sugar or a sugar alcohol (e.g., mannitol, isomalt, sorbitol, dextrose, xylitol, sucrose and lactose); the filler comprises particles having an average diameter between 50 μm and 1000 μm; the lubricant and/or glidant is selected from: talc, leucine, magnesium stearate, stearic acid and polyvinyl alcohol; and the lubricant and/or glidant is selected from: calcium stearate, mineral oil, vegetable oil, PEG (e.g., PEG that is liquid or solid at room temperature), sodium benzoate, and sodium lauryl sulfate.

In some cases, the therapeutic polypeptide solution used in a method for preparing the formulation has a pH below 7 (e.g., a pH between 1 and 3 or a pH between about 1.5 and about 2.5). The pH can be adjusted with, e.g., phosphoric acid. In some cases, the solution is buffered. Various pharmaceutically acceptable buffers can be used (e.g., phosphate buffer).

In some cases, the therapeutic polypeptide solution used in a method for preparing the formulation comprises both a cation (e.g., CaCl₂) and a sterically hindered primary amine (e.g., leucine).

In some cases the therapeutic polypeptide solution comprises CaCl₂ and leucine; the binder is methylcellulose; the filler is microcrystalline cellulose; the glidant and/or lubricant comprises talc or leucine.

In certain embodiments the therapeutic polypeptide does not comprise or consist of the amino acid sequence CCEYCCNPACTGCY. In certain embodiments, the therapeutic polypeptide does not comprise or consist of a GC-C receptors agonist polypeptide.

Also featured is a pharmaceutical composition prepared by any of the methods described herein.

### DETAILED DESCRIPTION

Compositions containing a therapeutic polypeptide can include any therapeutic polypeptide, for example, which include, but are not limited to, bone morphogenic proteins, insulin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, bovine somatotropin, porcine somatotropin, oxytocin, vasopressin, GRF, somatostatin, lypressin, pancreozymin, luteinizing hormone, LHRH, LHRH agonists and antagonists, leuprolide, interferons such as interferon alpha-2a, interferon alpha-2b, and consensus interferon, interleukins, growth hormones such as human growth hormone and its derivatives such as methione-human growth hormone and des-phenylalanine human growth hormone, parathyroid hormone, bovine growth hormone and porcine growth hormone, fertility inhibitors such as the prostaglandins, fertility promoters, growth factors such as epidermal growth factors (EGF), platelet-derived growth factors (PDGF), fibro-blast growth factors (FGF), transforming growth factors-alpha (TGF-α), transforming growth factors-beta (TGF-β), erythropoietin (EPO), insulin-like growth factor-1-(IGF-I), insulin-like growth factor-II (IGF-II), interleukin-1, interleukin-2, interleukin-6, interleukin-8, tumor necrosis factor-alpha (TNF-α), tumor necrosis factor-beta (TNFβ), Interferon-alpha (INF-α), Interferon-beta (INF-β), Interferon-gamma (INF-γ), Interferon-omega (INF-Ω), colony stimulating factors (CSF), vascular cell growth factor (VEGF), thrombopoietin (TPO), stromal cell-derived factors (SDF), placenta growth factor (PIGF), hepatocyte growth factor (HGF), granulocyte macrophage colony stimulating factor (GM-CSF), glial-derived neurotropin factor (GDNF), granulocyte colony stimulating factor (G-CSF), ciliary neurotropic factor (CNTF), bone growth factor, transforming growth factor, bone morphogeneic proteins (BMP), coagulation factors, human pancreas hormone releasing factor, analogs and derivatives of these polypeptides, and pharmaceutically acceptable salts of these compounds, or their analogs or derivatives. In certain embodiments, the therapeutic polypeptide may be a mixture of two or more therapeutic polypeptides described herein.

In some embodiments, the solid, stable formulation of the therapeutic polypeptide is administered orally. In other embodiments, the solid, stable formulation is solubilized in an appropriate excipient for administration by other routes. For example, the formulation may be solubilized and the therapeutic polypeptide may be administered, e.g., by intravenous injection, intramuscular injection, subcutaneous injection, intraperitoneal injection, topical, sublingual, intraarticular (in the joints), intradermal, buccal, ophthalmic (including intraocular), intranasally (including using a cannula), intraspinally or intrathecally. In one embodiment, the therapeutic polypeptide composition is provided in a discrete unit, a unit dosage form, (e.g., a tablet, a capsule, a sachet) that is effective at such dosage either for administration orally or for solubilization and subsequent administration by other routes. In another embodiment, the therapeutic polypeptide is provided in a unit dosage form either for administration orally or for solubilization for subsequent administration by other routes, wherein the unit dosage form provides multiple effective dosages (i.e., each unit dosage form provides more than one effective dosages of the therapeutic polypeptide). In another embodiment, the therapeutic polypeptide is provided in a unit dosage form that provides an effective dosage with multiple unit dosage forms either for administration orally or for solubilization and subsequent administration by other routes. In certain embodiments, the unit dosage form and daily dose are equivalent. In various embodiments, the unit dosage form is administered orally with food at anytime of the day, without food at anytime of the day, with food after an overnight fast (e.g. with breakfast). In various embodiments, the unit dosage form is administered once a day, twice a day or three times a day either orally or via another route. In various embodiments, the unit dosage form is administered once a week, twice a week, three times a week, once every two weeks, once every three weeks, once every four weeks, once a month, once every two months, once every three months, or once every six months either orally or via another route. The unit dosage form can optionally comprise other additives. In some embodiments, one, two or three unit dosage forms will contain the dose of therapeutic polypeptide. The precise amount of compound administered to a patient will be the responsibility of the attendant physician. However, the dose employed will depend on a number of factors, including the age and sex of the patient, the precise disorder being treated, and its severity.

A cation of the invention may be provided as a pharmaceutically acceptable salt i.e., a cation with an appropriate counterion. Examples of pharmaceutically acceptable salts that may be used in the invention include, without limitation, magnesium acetate, magnesium chloride, magnesium phosphate, magnesium sulfate, calcium acetate, calcium chloride, calcium phosphate, calcium carbonate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate. In one embodiment, a pharmaceutically acceptable salt that may be used is calcium chloride, magnesium chloride and zinc acetate.

As used herein, the sterically hindered primary amine has the formula

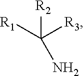

wherein R₁, R₂ and R₃ are independently selected from: H; —C(O)OH; C₁-C₆ alkyl, optionally substituted by —CO₂H, —CONH₂, or a 5-10 membered aryl or heteroaryl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

The term “alkyl”, as used herein, refers to a saturated linear or branched-chain monovalent hydrocarbon radical. Unless otherwise specified, an alkyl group contains 1-20 carbon atoms (e.g., 1-20 carbon atoms, 1-10 carbon atoms, 1-8 carbon atoms, 1-6 carbon atoms, 1-4 carbon atoms or 1-3 carbon atoms). Examples of alkyl groups include, but are not limited to, methyl, ethyl, n-propyl, isopropyl, n-butyl, isobutyl, s-butyl, t-butyl, pentyl, hexyl, heptyl, octyl and the like.

The terms Cₙ₋ₘ “alkoxyalkyl” and Cₙ₋ₘ “thioalkoxyalkyl” mean alkyl, substituted with one or more alkoxy or thioalkoxy groups, as the case may be, wherein the combined total number of carbons of the alkyl and alkoxy groups, or alkyl and thioalkoxy groups, combined, as the case may be, is between the values of n and m. For example, a C₄₋₆ alkoxyalkyl has a total of 4-6 carbons divided between the alkyl and alkoxy portion; e.g. it can be —CH₂OCH₂CH₂CH₃, —CH₂CH₂OCH₂CH₃ or —CH₂CH₂CH₂OCH₃.

As used herein, the term “aryl” (as in “aryl ring” or “aryl group”), used alone or as part of a larger moiety, refers to a carbocyclic ring system wherein at least one ring in the system is aromatic and has a single point of attachment to the rest of the molecule. Unless otherwise specified, an aryl group may be monocyclic, bicyclic or tricyclic and contain 6-18 ring members. Examples of aryl rings include, but are not limited to, phenyl, naphthyl, indanyl, indenyl, tetralin, fluorenyl, and anthracenyl.

The term “heteroaryl” (or “heteroaromatic” or “heteroaryl group” or “aromatic heterocycle”) used alone or as part of a larger moiety as in “heteroaralkyl” or “heteroarylalkoxy” refers to a ring system wherein at least one ring in the system is aromatic and contains one or more heteroatoms, wherein each ring in the system contains 3 to 7 ring members and which has a single point of attachment to the rest of the molecule. Unless otherwise specified, a heteroaryl ring system may be monocyclic, bicyclic or tricyclic and have a total of five to fourteen ring members. In one embodiment, all rings in a heteroaryl system are aromatic. Also included in this definition are heteroaryl radicals where the heteroaryl ring is fused with one or more aromatic or non-aromatic carbocyclic or heterocyclic rings, or combinations thereof, as long as the radical or point of attachment is in the heteroaryl ring. Bicyclic 6,5 heteroaromatic system, as used herein, for example, is a six membered heteroaromatic ring fused to a second five membered ring wherein the radical or point of attachment is on the six membered ring.

Heteroaryl rings include, but are not limited to the following monocycles: 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, 1,3,5-triazinyl, and the following bicycles: benzimidazolyl, benzofuryl, benzothiophenyl, benzopyrazinyl, benzopyranonyl, indolyl (e.g., 2-indolyl), purinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, the term “binder” refers to any pharmaceutically acceptable binder that may be used in the practice of the invention. Examples of pharmaceutically acceptable binders include, without limitation, corn starch, potato starch, other starches, gelatin, natural and synthetic gums such as acacia, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone (e.g., polyvinyl pyrrolidone K30), methyl cellulose, pre-gelatinized starch (e.g., STARCH 1500® and STARCH 1500 LM®, sold by Colorcon, Ltd.), hypromellose (hydroxypropyl methylcellulose), microcrystalline cellulose (e.g. AVICEL™, such as, AVICEL-PH-101™, -103™ and -105™, sold by FMC Corporation, Marcus Hook, Pa., USA), and mixtures thereof.

As used herein, the term “filler” refers to any pharmaceutically acceptable filler that may be used in the practice of the invention. Examples of pharmaceutically acceptable fillers include, without limitation, talc, calcium carbonate (e.g., granules or powder), dibasic calcium phosphate, tribasic calcium phosphate, calcium sulfate (e.g., granules or powder), microcrystalline cellulose (e.g., Avicel PH101), powdered cellulose, dextrates, kaolin, mannitol, silicic acid, sorbitol, starch, pre-gelatinized starch, lactose, glucose, fructose, galactose, trehalose, sucrose, maltose, raffinose, maltitol, melezitose, stachyose, lactitol, palatinite, xylitol, mannitol, myoinositol, and mixtures thereof.

Examples of pharmaceutically acceptable fillers that may be particularly used for coating with therapeutic polypeptide include, without limitation, talc, microcrystalline cellulose (e.g., Avicel PH101), powdered cellulose, dextrates, kaolin, mannitol, silicic acid, sorbitol, starch, pre-gelatinized starch, lactose, glucose, fructose, galactose, trehalose, sucrose, maltose, isomalt, dibasic calcium phosphate, raffinose, maltitol, melezitose, stachyose, lactitol, palatinite, xylitol, mannitol, myoinositol, and mixtures thereof.

As used herein, the term “additives” refers to any pharmaceutically acceptable additive. Pharmaceutically acceptable additives include, without limitation, disintegrants, dispersing additives, lubricants, glidants, antioxidants, coating additives, diluents, surfactants, flavoring additives, humectants, absorption promoting additives, controlled release additives, anti-caking additives, anti-microbial agents (e.g., preservatives), colorants, desiccants, plasticizers and dyes.

As used herein, an “excipient” is any pharmaceutically acceptable additive, filler, binder or agent.

As used herein, “purified therapeutic polypeptide” is therapeutic polypeptide or a pharmaceutically acceptable salt thereof that is greater than or equal to 95 percent pure. therapeutic polypeptide purity can be measured, for example, by chromatographic purity of therapeutic polypeptide using HPLC.

In some embodiments, the therapeutic polypeptide composition is provided in a solid form for oral administration. Examples of such forms include, without limitation, a tablet, a sachet, a pellet, a capsule or a powder. In some embodiments, the compositions can be used to create unit dosages forms, e.g., tablets, capsules, sachets or pellets. Orally administered compositions can include, for example, binders, lubricants, inert diluents, lubricating, surface active or dispersing additives, flavoring additives, and humectants. Orally administered formulations such as tablets may optionally be coated or scored and may be formulated so as to provide sustained, delayed or controlled release of the therapeutic polypeptide therein. The therapeutic polypeptide can be co-administered or co-formulated with other medications.

The compositions can include, for example, various additional solvents, dispersants, coatings, absorption promoting additives, controlled release additives, and one or more inert additives (which include, for example, starches, polyols, granulating additives, microcrystalline cellulose, diluents, lubricants, binders, disintegrating additives, and the like), etc. If desired, tablet dosages of the disclosed compositions may be coated by standard aqueous or non-aqueous techniques. Compositions can also include, for example, anti-caking additives, preservatives, sweetening additives, colorants, flavors, desiccants, plasticizers, dyes, and the like.

Suitable disintegrants include, for example, agar-agar, calcium carbonate, microcrystalline cellulose, croscarmellose sodium, crospovidone, povidone, polacrilin potassium, sodium starch glycolate, potato or tapioca starch, other starches, pre-gelatinized starch, clays, other algins, other celluloses, gums, and mixtures thereof.

Suitable lubricants include, for example, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Evonik Degussa Co., Plano, Tex. USA), a pyrogenic silicon dioxide (CAB-O-SIL, Cabot Co., Boston, Mass. USA), and mixtures thereof.

Suitable anti-caking additives include, for example, calcium silicate, magnesium silicate, silicon dioxide, colloidal silicon dioxide, talc, and mixtures thereof.

Suitable anti-microbial additives that may be used, e.g., as a preservative for the therapeutic polypeptide compositions, include, for example, benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, and mixtures thereof.

Suitable coating additives include, for example, sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, carnauba wax, microcrystalline wax, and mixtures thereof.

In certain embodiments, suitable additives for the therapeutic polypeptide composition include one or more of sucrose, talc, magnesium stearate, crospovidone or BHA.

In certain embodiments, the term “95%” may be 95.0%, the term “90%” may be 90.0%, the term “10%” may be 10.0%, the term “9%” may be 9.0%, the term “8%” may be 8.0%, the term “7%” may be 7.0%, the term “6%” may be 6.0%, the term “5%” may be 5.0%, the term “4%” may be 4.0%, the term “3%” may be 3.0%, the term “2%” may be 2.0%, and the term “1%” may be 1.0%.

In certain embodiments, the therapeutic polypeptide composition is provided in a unit dosage form. In some embodiments, the unit dosage form is a capsule, a tablet, a sachet, a pellet or a powder. In one such embodiment, the unit dosage form is a capsule or tablet. Such unit dosage forms may be contained in a container such as, without limitation, a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to provide a single dosage form. For example, tablets or capsules may be contained in a bottle which is in turn contained within a box. In some embodiments, the unit dosage forms are provided in a container further comprising a desiccant. In a further embodiment, the unit dosage forms, e.g., a quantity of tablets or capsules, are provided in a container, e.g., a bottle, jar or re-sealable bag, containing a desiccant. In a further embodiment, the container containing the unit dosage forms is packaged with administration or dosage instructions. In certain embodiments, the therapeutic polypeptide composition is provided in a kit. The therapeutic polypeptide composition described herein and combination therapy agents can be packaged as a kit that includes single or multiple doses of two or more agents, each packaged or formulated individually, or single or multiple doses of two or more agents packaged or formulated in combination. Thus, the therapeutic polypeptide composition can be present in first container, and the kit can optionally include one or more agents in a second container. The container or containers are placed within a package, and the package can optionally include administration or dosage instructions.

### EXAMPLES

A therapeutic polypeptide or a pharmaceutically acceptable salt thereof may be produced and purified using standard techniques known in the art, e.g., chemical synthesis or recombinant expression followed by and purification using standard techniques.

### Example 1

### Formulation Method A

Preparation of the Coating Solution: Approximately 32 g to 42 g of purified water is mixed with hydrochloric acid to create a solution with a pH between 1.5 and 2.0. The cation, if used, is added to the solution in a quantity to provide the desired concentration, and the solution is mixed for sufficient time to produce a clear solution. The sterically hindered primary amine, if used, is added to the solution in a quantity to provide the desired concentration, and the solution is mixed for sufficient time to produce a clear solution. Other additives, such as antioxidants, are then added, if desired. The pH of the solution is tested, and hydrochloric acid is added, if necessary, to produce a solution having a pH between 1.5 and 2.0. The binder is then added to the solution and the mixture is then stirred for sufficient time to achieve a clear solution. The desired amount of therapeutic polypeptide is added to the solution and mixed for 30-100 minutes to provide the coating solution.

Preparation of the Active Beads: Approximately 30-36 g of dried microcrystalline cellulose beads are added to a Mini Column Fluid Bed Coater. The microcrystalline cellulose beads are fluidized and heated prior to layering. Next, the coating solution is layered to the beads. The spraying temperature is controlled between 24° C. and 55° C. by controlling inlet temperature, spray rate, atomization pressure, and air volume. After the entire coating solution is layered to the beads, the beads are dried. The product of this process is referred to as active beads.

### Example 2

### Formulation Method B

Preparation of the Coating Solution: Approximately 8.3 kg of purified water is mixed with hydrochloric acid to create a solution with a pH between 1.5 and 2.0. The cation, if used, is added to the solution in a quantity to provide the desired concentration, and the solution is mixed for sufficient time to produce a clear solution. The sterically hindered primary amine, if used, is added to the solution in a quantity to provide the desired concentration, and the solution is mixed for sufficient time to produce a clear solution. Other additives, such as antioxidants, are then added, if desired. The binder is then added to the solution and the solution is mixed for sufficient time to achieve a clear solution. The pH of the solution is tested, and hydrochloric acid is added if necessary to produce a solution having a pH between 1.5 and 2.0. This is Solution 1. Approximately 8.3 kg of purified water is mixed with hydrochloric acid to create a solution with a pH between 1.5 and 2.0. The desired amount of therapeutic polypeptide is added to the solution and mixed for 10 to 30 minutes. The pH of the solution is tested, and hydrochloric acid is added if necessary to produce a solution having a pH between 1.5 and 2.0. This is Solution 2. Solution 1 and Solution 2 are then mixed together. The pH of the solution is tested, and hydrochloric acid is added if necessary to produce a solution having a pH between 1.5 and 2.0. This is the coating solution.

Preparation of the Active Beads: Approximately 24.19 kg of microcrystalline cellulose beads are added to a Wurster Column of a Glatt GPCG-30 Fluid Bed. The microcrystalline cellulose beads are fluidized and heated to product temperature of 45-47° C. Next, the coating solution is layered to the beads. The product spraying temperature is controlled between 37° C. and 47° C. by controlling inlet temperature, spray rate, atomization pressure, and air volume. After the entire coating solution is layered to the beads, the beads are dried with a product drying temperature of 37° C. to 47° C. The product of this process is referred to as active beads.

### Example 3

### Preparation of Capsules Containing a Therapeutic Polypeptide Formulation

The therapeutic polypeptide content on active beads may be measured as described below or by other equivalent methods.

To form capsules suitable for oral administration, an appropriate amount of active beads is used to fill gelatin capsules (e.g., Size 2 gelatin capsules). An appropriate amount of active beads may contain 50 μg to 2 mg therapeutic polypeptide per capsule with a range of ±5%. In another embodiment, an appropriate amount of active beads to fill a desired number of gelatin capsules is placed in a container. One or more pharmaceutically acceptable fillers or other pharmaceutically acceptable additives may be added, if desired, to the container. In some embodiments, a filler or additive is talc, leucine, microcrystalline cellulose or mannitol. The contents of the container are blended and the mixture is used to fill gelatin capsules with an appropriate amount of active beads containing therapeutic polypeptide (e.g., 50 μg to 2 mg therapeutic polypeptide per capsule with a range of ±5%).

In an alternative embodiment, an appropriate amount of active beads is used to fill gelatin capsules and one or more pharmaceutically acceptable fillers or other pharmaceutically acceptable additives are added to the gelatin capsules.

### Example 5

### Measurement of Therapeutic Polypeptide Content and Purity

Therapeutic polypeptide content and purity may be determined by reverse phase gradient liquid chromatography. The therapeutic polypeptide content is measured by determining the therapeutic polypeptide concentration in the prepared sample against a similarly prepared external therapeutic polypeptide standard.

1-157. (canceled)

158. A pharmaceutical composition comprising a therapeutic polypeptide and a pharmaceutically acceptable excipient, wherein
(i) the chromatographic purity of the therapeutic polypeptide decreases by less than 10% after (a) 18 months of storage of the pharmaceutical composition at 25° C. at 60% relative humidity in a sealed container containing a desiccant or (b) 6 months of storage of the pharmaceutical composition at 40° C. at 75% relative humidity in a sealed container containing a desiccant;
(ii) wherein the chromatographic purity of the therapeutic polypeptide is greater than or equal to 90% after (a) 18 months of storage of the pharmaceutical composition at 25° C. at 60% relative humidity in a sealed container containing a desiccant or (b) 6 months of storage of the pharmaceutical composition at 40° C. at 75% relative humidity in a sealed container containing a desiccant;
(iii) the assay value for therapeutic polypeptide determined on a weight/weight basis decreases by less than 10% after (a) 18 months of storage of the pharmaceutical composition at 25° C. at 60% relative humidity in a sealed container containing a desiccant or (b) 6 months of storage of the pharmaceutical composition at 40° C. at 75% relative humidity in a sealed container containing a desiccant; or
(iv) the assay value for therapeutic polypeptide determined on a weight/weight basis is greater than or equal to 90% after (a) 18 months of storage of the pharmaceutical composition at 25° C. at 60% relative humidity in a sealed container containing a desiccant or (b) 6 months of storage of the pharmaceutical composition at 40° C. at 75% relative humidity in a sealed container containing a desiccant.

159. A pharmaceutical composition comprising:
a pharmaceutically acceptable carrier;
a therapeutic polypeptide; and
one or more agents selected from (i) a cation selected from Mg²⁺, Ca²⁺, Zn²⁺, Mn²⁺, K⁺, Na⁺ or Al³⁺or (ii) a sterically hindered primary amine,
wherein the agent improves at least one attribute of the composition, relative to a pharmaceutical composition without the agent, after (a) a first 18 months of storage of the pharmaceutical composition at 25° C. at 60% relative humidity in a sealed container containing a desiccant or (b) a first 6 months of storage of the pharmaceutical composition at 40° C. at 75% relative humidity in a sealed container containing a desiccant, and
wherein the attribute is selected from: a decrease in the rate of degradation of therapeutic polypeptide as measured by therapeutic polypeptide content, a decrease in the rate of degradation of therapeutic polypeptide as measured by chromatographic purity of therapeutic polypeptide, a decrease in the amount of a therapeutic polypeptide oxidation product relative to the amount of therapeutic polypeptide, and a decrease in the amount of a therapeutic polypeptide hydrolysis product relative to the amount of therapeutic polypeptide.

160. The pharmaceutical composition according to claim 159, wherein the agent is a cation selected from Mg²⁺, Ca²⁺, Zn²⁺, Mn²⁺, K⁺, Na⁺ or Al³⁺and is provided as magnesium acetate, magnesium chloride, magnesium phosphate, magnesium sulfate, calcium acetate, calcium chloride, calcium phosphate, calcium carbonate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate.

161. The pharmaceutical composition according to claim 160, wherein the cation is Ca²⁺and is provided as calcium chloride.

162. The pharmaceutical composition according to claim 159, wherein the agent is a sterically hindered primary amine selected from an amino acid or a compound of the formula: 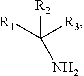
wherein R₁, R₂ and R₃ are independently selected from: H; —C(O)OH; C₁-C₆ alkyl, optionally substituted by —CO₂H, —CONH₂, or a 5-10 membered aryl or heteroaryl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H.

163. The pharmaceutical composition according to claim 162, wherein the sterically hindered primary amine is a naturally-occurring amino acid selected from the group consisting of histidine, phenylalanine, alanine, glutamic acid, aspartic acid, glutamine, leucine, methionine, asparagine, tyrosine, threonine, isoleucine, tryptophan and valine.

164. The pharmaceutical composition according to claim 163, wherein the naturally-occurring amino acid is leucine.

165. The pharmaceutical composition according to claim 159, comprising a cation selected from Mg²⁺, Ca²⁺, Zn²⁺, Mn²⁺, K⁺, Na⁺or Al³⁺and a sterically hindered primary amine.

166. A pharmaceutical composition comprising:
a pharmaceutically acceptable carrier;
a therapeutic polypeptide;
a cation selected from Mg²⁺, Ca²⁺, Zn²⁺, Mn²⁺, K⁺, Na⁺or Al³⁺; and
a sterically hindered primary amine.

167. The pharmaceutical composition according to claim 166, wherein the cation Mg²⁺, Ca²⁺, Zn²⁺, Mn²⁺, K⁺, Na⁺or Al³⁺is provided as magnesium acetate, magnesium chloride, magnesium phosphate, magnesium sulfate, calcium acetate, calcium chloride, calcium phosphate, calcium carbonate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate.

168. The pharmaceutical composition according to claim 167, wherein the cation is Ca²⁺and is provided as calcium chloride.

169. The pharmaceutical composition according to claim 166, wherein the sterically hindered primary amine is selected from an amino acid or a compound of the formula: 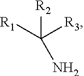
wherein R₁, R₂ and R₃ are independently selected from: H; —C(O)OH; C₁-C₆ alkyl, optionally substituted by —CO₂H, —CONH₂, or a 5-10 membered aryl or heteroaryl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H.

170. The pharmaceutical composition according to claim 169, wherein the sterically hindered primary amine is a naturally-occurring amino acid selected from the group consisting of histidine, phenylalanine, alanine, glutamic acid, aspartic acid, glutamine, leucine, methionine, asparagine, tyrosine, threonine, isoleucine, tryptophan and valine.

171. The pharmaceutical composition according to claim 170, wherein the naturally-occurring amino acid is leucine.

172. The pharmaceutical composition according to claim 169, wherein the sterically hindered primary amine is a non-naturally occurring amino acid selected from 1-aminocyclohexane carboxylic acid, cyclohexylamine, and 2-methylbutylamine.

173. The pharmaceutical composition according to claim 169, wherein the sterically hindered primary amine is chitosan.

174. The pharmaceutical composition according to claim 166, wherein the cation is provided as Ca²⁺and the sterically hindered primary amine is leucine, and the molar ratio of Ca²⁺to leucine is at least 1:1, 1.5:1, or 2:1.

175. The pharmaceutical composition according to claim 166, wherein the sterically hindered amine is leucine and the molar ratio of leucine to the therapeutic polypeptide is at least 10:1, 20:1, or 30:1.

176. The pharmaceutical composition according to claim 166, wherein the molar ratio of cation:sterically hindered primary amine:therapeutic polypeptide is 40-100:20-50:1.

177. The pharmaceutical composition according to claim 166, wherein the cation is Ca²⁺, the sterically hindered primary amine is leucine, and the molar ratio of Ca²⁺:leucine:therapeutic polypeptide is 100:30:1, 80:40:1, 80:30:1, 80:20:1, 60:30:1, 60:20:1, 50:30:1, 50:20:1, 40:20:1, 20:20:1, 10:10:1, 10:5:1, 5:10:1 or 5:5:1.

178. The pharmaceutical composition according to claim 177, wherein the molar ratio of Ca²⁺:leucine:therapeutic polypeptide is 60:30:1.

179. The pharmaceutical composition according to claim 166, further comprising one or more of a pharmaceutically acceptable binder, a pharmaceutically acceptable glidant, lubricant or additive that acts as both a glidant and lubricant, an antioxidant, or a pharmaceutically acceptable filler.

180. The pharmaceutical composition according to claim 179, wherein
the antioxidant, when present, is BHA, vitamin E or propyl gallate;
the pharmaceutically acceptable binder, when present, is selected from polyvinyl alcohol, polyvinylpyrrolidone (povidone), a starch, maltodextrin or a cellulose ether; and
the pharmaceutically acceptable filler, when present, is selected from cellulose, isomalt, mannitol or dibasic calcium phosphate.

181. The pharmaceutical composition according to claim 180, wherein
the cellulose ether, when present, is selected from methylcellulose, ethylcellulose, carboxymethylcellulose, hydroxyethyl cellulose, hydroxyethyl methylcellulose, hydroxypropyl cellulose and hydroxypropyl methylcellulose; and
the cellulose, when present, is selected from microfine cellulose and microcrystalline cellulose.

182. The pharmaceutical composition according to claim 179, wherein the pharmaceutically acceptable filler comprises particles having an average diameter between 150 μm and 1000 μm.

183. The pharmaceutical composition according to claim 179, wherein the pharmaceutical composition comprises a pharmaceutically acceptable filler and the weight ratio of the therapeutic polypeptide to pharmaceutically acceptable filler is between 1:25 and 1:2,500; between 1:100 and 1:2000; or between 1:100 and 1:1000.

184. A capsule or tablet comprising the pharmaceutical composition according to claim 166.

185. The capsule or tablet according to claim 184, wherein each capsule or tablet comprises 25 μg to 1 g of the therapeutic polypeptide.

186. A method for preparing a pharmaceutical composition comprising a therapeutic polypeptide or a salt thereof, the method comprising:
(a) providing an aqueous solution comprising:
(i) a therapeutic polypeptide or a pharmaceutically acceptable salt thereof
(ii) one or more of a cation selected from Mg²⁺, Ca²⁺, Zn²⁺, Mn²⁺, K⁺, Na⁺ or Al³⁺and a sterically hindered primary amine; and
(iii) a pharmaceutically acceptable binder; and
(b) applying the aqueous solution to a pharmaceutically acceptable filler to generate therapeutic polypeptide-coated filler.

187. The method according to claim 186, wherein the aqueous solution comprises a cation and a sterically hindered primary amine.

188. The method according to claim 186, wherein the aqueous solution is applied to the filler by spraying

189. The method according to claim 186, further comprising tableting or encapsulating the therapeutic polypeptide-coated filler in a tablet or capsule, respectively.

190. The method according to claim 189, wherein the capsule is a gelatin capsule

191. The method according to claim 190, wherein the capsule contains 25 μg to 1 g the therapeutic polypeptide.
